(19) United States
(12) Patent Application Publication
(10) Pub. No.: US 2024/0217899 A1
(43) Pub. Date: Jul. 4, 2024

(54) PROCESS FOR PRODUCING RENEWABLE PRODUCT STREAMS

(71) Applicant: UOP LLC, Des Plaines, IL (US)

(72) Inventors: Avram M. Buchbinder, Chicago, IL (US); Stanley Joseph Frey, Palatine, IL (US); Zhanyong LI, Schaumburg, IL (US); Krishna Mani, Gurgaon (IN); Richa Sharma, Gurgaon (IN); Krishan Pratap Jadaun, Gurugram (IN); Eseoghene Jeroro, Chicago, IL (US)

(21) Appl. No.: 18/394,840

(22) Filed: Dec. 22, 2023

Related U.S. Application Data
(60) Provisional application No. 63/436,508, filed on Dec. 31, 2022.

(51) Int. Cl. C07C 4/06; C07C 7/12

(52) U.S. Cl. CPC C07C 4/06; C07C 7/12; C07C 2523/28; C07C 2523/42; C07C 2523/755

(57) ABSTRACT

A biorenewable feed that is concentrated in free fatty acids is produced by hydrodeoxygenating a biorenewable feedstock is produced by use of a Group VIII catalyst producing a 10-13 carbon atom product having a high level of linearity. Normal paraffins in the range desired by the detergents industry can be produced. Either isomerization or an iso-normal separation can be performed to provide green fuel streams.

This application claims priority to provisional application 63/436,508, filed Dec. 31, 2022.

### FIELD

The field is processes for producing product streams from renewable feed streams. Specifically, the field is processes for producing detergent streams and fuel streams from renewable feed streams.

### BACKGROUND

Linear alkylbenzenes are organic compounds with the formula C₆H₅CHₙH₂ₙ₊₁. While the alkyl carbon number, “n” can have any practical value, detergent manufacturers desire that alkylbenzenes have alkyl carbon number in the range of 9 to 16 and preferably in the range of 10 to 13. These specific ranges are often required when the alkylbenzenes are used as intermediates in the production of surfactants for detergents. The alkyl carbon number in the range of 10 to 13 falls in line with the specifications of the detergents industry.

Because the surfactants created from alkylbenzenes are biodegradable, the production of alkylbenzenes has grown rapidly since their initial uses in detergent production in the 1960s. The linearity of the paraffin chain in the alkylbenzenes is key to the material's biodegradability and effectiveness as a detergent. A major factor in the final linearity of the alkylbenzenes is the linearity of the paraffin component.

While detergents made utilizing alkylbenzene-based surfactants are biodegradable, previous processes for creating alkylbenzenes are not based on renewable sources. Specifically, alkylbenzenes are currently produced from kerosene refined from crude extracted from the earth. Due to the growing environmental prejudice against fossil fuel extraction and economic concerns over exhausting fossil fuel deposits, there may be support for using an alternate source for biodegradable surfactants in detergents and in other industries.

Accordingly, it is desirable to provide linear alkylbenzenes with a high degree of linearity that are made from biorenewable sources instead of being extracted from the earth. Further, it is desirable to provide renewable linear alkylbenzenes from easily processed triglycerides and fatty acids from vegetable, animal, nut, and/or seed oils. Palm kernel oil, coconut oil and babassu oil have a composition that is high in the desirable range of C₁₀-C₁₃ n-paraffins that aligns with the alkyl carbon number range desired of the detergent industry. Most other renewable triglyceride sources have a high amount of nC₁₆ to nC₁₈ content and it is desirable to be able to convert those feeds to nC₁₀ to nC₁₃ feeds with a high per-pass yield. These nC₁₀ to nC₁₃ intermediate products are useful in eventually making linear alkylbenzene types of detergents through additional process steps. It is further desirable that the resulting nC₁₀ to nC₁₃ paraffins are linear products with a minimum of branched isomer products.

Biofuels may be co-produced with linear alkylbenzenes. Other desirable features and characteristics of the present invention will become apparent from the subsequent detailed description of the invention and the appended claims, when taken in conjunction with the accompanying drawing and this background.

### BRIEF SUMMARY

We have discovered that feeds containing longer normal paraffins with 14-22 carbon atoms, derived from biorenewable feeds such as fats oils and greases with high content of triglycerides, can be converted to paraffin compositions containing normal paraffins with 10-13 carbon atoms favored in detergent alkylation, by use of a preferred catalyst such as a supported group VIII metal catalyst, e.g. Ru—ZrO₂ or Pt—Al₂O₃ or Ni—ZrO₂ or a Mo-based catalyst such as Mo on alumina. The per-pass nC₁₀ to nCt₃ yield from the new catalyst and process can be significantly higher (˜30%) than what one can obtain from the prior art process (˜5%). It was found that depending upon the catalyst that is used that the yield of the desired C₁₀-C₁₃ may vary, the linearity of the C₁₀-C₁₃ with normal hydrocarbons preferred and the amount of methane produced as a byproduct.

Additional details and embodiments of the disclosure will become apparent from the following detailed description of the disclosure.

### BRIEF DESCRIPTION OF THE DRAWINGS

FIG. 1 is a schematic view of a conversion unit of the present disclosure;

FIG. 2 is a schematic view of an alternate conversion unit of FIG. 1; and

FIG. 3 is a schematic view of a benzene alkylation unit useful with the conversion unit of either FIG. 1 or FIG. 2.

### DETAILED DESCRIPTION

The present disclosure endeavors to produce alkylbenzenes for detergent production and jet fuel and/or diesel from renewable sources. The raw materials for the generation of linear alkyl benzenes are normal C₁₀ to C₁₃ olefins, which can be generated by dehydrogenation of normal C1₀-C₁₃ paraffins. Normal paraffins with 10 to 13 carbons are the desired number of carbons that detergent producers desire for the addition of the alkyl group on the alkylbenzenes used in detergents. While current processes generally use fossil sources of normal paraffins such as kerosene, there is a need for bio-renewable sources of normal paraffins. The most available source of bio-renewable n-paraffins is to subject a stream containing triglycerides or fatty acids to a deoxygenation process such as hydrodeoxygenation to generate an intermediate stream of normal paraffins. However, most triglyceride and fatty acid containing feedstocks produce normal paraffins with 16 to 18 carbons, and in some cases 22 carbons, when deoxygenated. These normal paraffins are of longer chain length that desired by detergent producers. Some renewable sources such as palm kernel oil (PKO), coconut oil and babassu oil have fatty acids that produce normal paraffins with 10 to 13 carbons when deoxygenated, so one path to generate C₁₀ to C3 normal paraffins is to use one of these triglyceride containing feeds. In a broad description, it has been found desirable to be able to take an intermediate stream containing broad range of C₁₆-C₂₂ normal paraffins that was generated from readily available fats, oils and greases (FOGS) to produce a desired product stream of normal C₁₀-C₁₃ by a hydrocracking-like process. The intermediate feed stream is treated as necessary to remove sulfur which deactivates the catalyst. A difference between the catalysts typically used for hydrotreating and for instant the hydrocracking-like step is that the hydrocracking catalysts have been reduced rather than sulfided.

We have found that the selection of particular metal catalysts can produce a much higher yield of normal paraffins with 10-13 carbons from a stream containing normal paraffins of 14 to 22 carbons than in previous processes. Compared to the traditional LAB process where the feed is from petroleum, the feed for this process starts with nC₁₆-nC₁₈ hydrocarbons that are from renewable sources such as soybean oil, corn oil and other fats, oils, and greases from biological sources. This renewable n-paraffin feed is generally obtained by hydrodeoxygenation of triglycerides in a process such as Ecofining by UOP LLC, Des Plaines, IL. With the catalyst that is used herein, it has been found that the nC₁₆-C₁₈ intermediate stream is able to generate linear cracking products with low amounts of branched isomer production.

Of the preferred catalysts, the Ru catalyst exhibits much higher activity and per-pass nC₁₀ to nC₁₃ yield than the other catalysts. Under the optimized reaction conditions, it also produces very small amounts of methane and isomerized product. This has been found to be the best catalyst for such chemical transformation process. The Pt—Al₂O₃ catalyst can produce even lower methane yield than the Ru based catalyst with slightly less linear product yield. However, it was found that the Mo containing catalysts produced high yield of the desired nC₁₀-nC₁₃ with a high degree of linearity and low amounts of methane.

To limit catalyst deactivation and poisoning, the feed is treated to remove sulfur contamination for the supported catalysts. The feed has less than 1 wt ppm sulfur content but for the purposes of this disclosure, a feed with less than 100 wt. ppm is considered to be sulfur free. Without this treatment, sulfur accumulates on the catalyst and leads to deactivation. A high temperature hydrogen treatment is shown to recover some of the lost activity.

The bio-renewable triglyceride containing feed is subjected to hydrodeoxygenation to generate the intermediate normal paraffin feed. The hydrodeoxygenation reactor reacts the bio-renewable triglyceride containing feed with hydrogen and converts triglycerides and free fatty acids to propane, water, n-paraffins and small amounts of ammonia, carbon monoxide and carbon dioxide. Generally, the n-paraffins are separated from the water and gaseous products in a separator to generate the intermediate normal paraffin stream. Hydrodeoxygenation should be complete or nearly complete such that the intermediate feed stream contains less than about 1000 ppm of oxygen, preferably less than 10 ppm of oxygen. The hydrodeoxygenation reactor temperatures are kept low, less than 343° C. (650° F.) for typical biorenewable feedstocks and less than 304° C. (580° F.) for feedstocks with higher free fatty acid (FFA) concentration to avoid polymerization of olefins found in FFA. Generally, hydrodeoxygenation reactor pressure of about 700 kPa (100 psig) to about 21 MPa (3000 psig) are suitable.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator which may be operated at higher pressure. The term “communication” means that fluid flow is operatively permitted between enumerated components, which may be characterized as “fluid communication”. The term “downstream communication” means that at least a portion of fluid flowing to the subject in downstream communication may operatively flow from the object with which it fluidly communicates.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Unless indicated otherwise, overhead lines and bottoms lines refer to the net lines from the column downstream of any reflux or reboil take-off to the column. Stripper columns may omit a reboiler at a bottom of the column and instead provide heating requirements and separation impetus from a fluidized inert media such as steam.

The catalysts that are used in the hydrocracking reactions are selected from a Ru on ZrO₂ catalyst, a Pt on Al₂O₃ catalyst, a Ni on alumina catalyst, Ni on ZrO₂ catalyst, NiO/clay catalyst, a Ni—Mo on alumina catalyst and a Mo catalyst that may be on alumina. The catalyst may be Ru—ZrO₂ (0.1 wt %)

Preferred cracking reaction conditions for the hydrocracking-like process include a temperature from about 230° C. (446° F.) to about 455° C. (850° F.), suitably 316° C. (600° F.) to about 427° C. (800° F.) and preferably 343° C. (650° F.) to about 399° C. (750° F.). For the Ru—ZrO2 catalyst, being the most active, suitable temperatures are lower than for the other catalysts; generally, about 230° C. (446° F.) to about 300° C. (572° F.). Suitable reaction pressure is from about 2.8 MPa (gauge) (400 psig) to about 17.5 MPa (gauge) (2500 psig), a liquid hourly space velocity of the fresh hydrocarbonaceous intermediate feed stream from about 0.1 hr⁻¹, suitably 0.5 hr⁻¹, to about 5 hr⁻¹, preferably from about 1.5 to about 4 hr⁻¹, and a hydrogen rate of about 84 Nm³/m³ (500 scf/bbl), to about 1,011 Nm³/m³ oil (6,000 scf/bbl), preferably about 168 Nm³/m³ oil (1,000 scf/bbl) to about 1,250 Nm³/m³ oil (7,500 scf/bbl). The cracking reactor is generally downstream of a reactor containing hydrotreating catalyst or a combination of hydrotreating catalysts.

As used herein, the term “a component-rich stream” or “a component stream” means that the stream coming out of a vessel has a greater concentration of the component than the feed to the vessel. As used herein, the term “a component-lean stream” means that the lean stream coming out of a vessel has a smaller concentration of the component than the feed to the vessel.

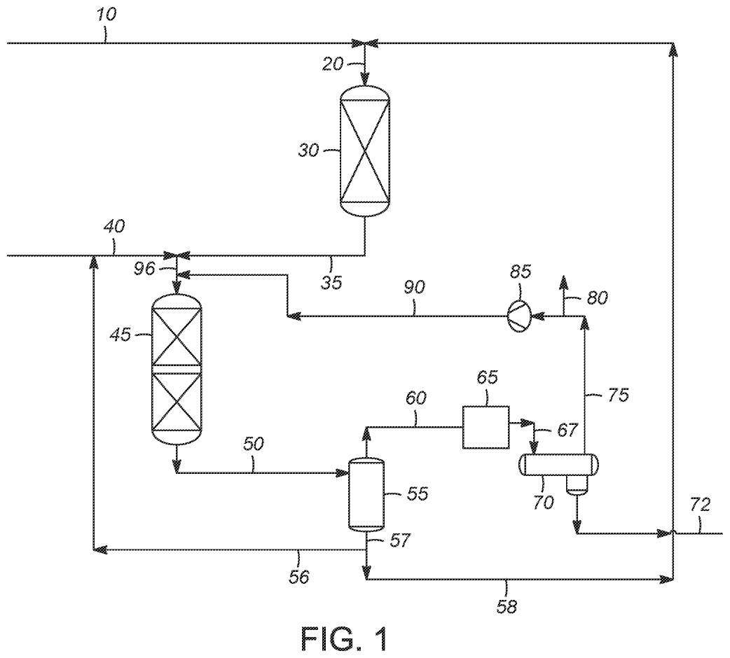

A basic process design showing a hydrotreating reactor and hydrocracking-like cracking reactor with the hydrocracking-like process in back-stage configuration is shown in FIG. 1 in which a feed 40 of a sustainable feedstock such as a soybean oil or corn oil that is rich in nC₁₆ to nC₂₂ are sent to be combined with a back stage effluent 35 from a hydrocracking-like cracking reactor 30 (henceforth referred to has hydrocracking reactor), containing one of the catalysts of the present invention, to be sent to a hydrotreating rector 45. The effluent 50 from the hydrotreating reactor 45 is sent to a separator 55 in which a liquid hydrocarbon stream 57 is split into a stream 56 to be combined with feed 40 and a stream 58 which is combined with a hydrogen stream 10 to be sent in stream 20 to hydrocracking reactor 30. An aqueous product 54 is collected in separator 55. An upper stream 60 is sent to vessel 65 to stream 67 and a 3-phase separator 70 to be split into stream 75 sent through compressor 85 to stream 90 which is combined with stream 96 to be sent to the hydrotreating reactor 45. A hydrocarbon stream 72 is sent to column 74 to be divided into off gas 79, LPG 78, LNAP 77 and HNAP 76.

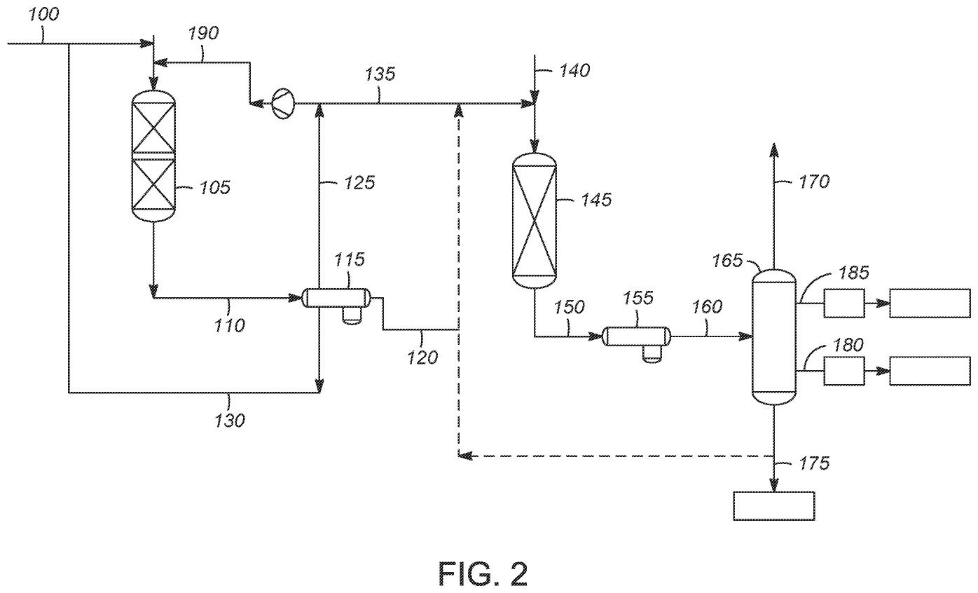

FIG. 2 shows an embodiment using a hydrotreating reactor and hydrocracking reactor in series using the catalysts of the present invention to produce a higher level of the desired C₁₀-C₁₃ carbons for use in making more linear alkyl benzene. A vegetable oil stream 100 is sent to a hydrotreating reactor 105 which contains hydrodeoxygenation catalysts. A stream of the hydrotreated hydrocarbon 110 is sent to separator 115 to produce a stream 130 to be recycled to vegetable oil stream 100, a stream 120 to be sent to a hydrocracking reactor 145, an aqueous product stream 11, and a gas stream 125 to be split, a portion of which is compressed to form stream 190 and recycled. The normal paraffin containing intermediate stream 120 is optionally combined with recycle product stream 175 and hydrogen stream 140 to form stream 139. Optionally, a portion of the recycle hydrogen stream 125 is treated to remove H₂S and NH₃ in treatment zone 137 and also combined with the paraffin containing intermediate stream 120. Stream 139 is passed to the hydrocracking reactor containing a reduced or partially reduced Ru, Pt, Ni or Mo containing catalyst in which the hydrocarbons are cracked into a hydrocarbon mixture 150 including normal paraffins. These hydrocarbons are sent to column 165 to be separated into an off-gas 170, a lighter hydrocarbon stream 185 to be sent to a steam cracker or otherwise utilized to make further products or fuels, and a stream 180 of C₁₀ to C₁₃ hydrocarbons which are then sent to be reacted to produce linear alkyl benzene product. Optionally a stream 175 is sent to an isomerization reactor to produce renewable fuels and a portion of stream 175 is recycled to be sent back through hydrocracking reactor 145.

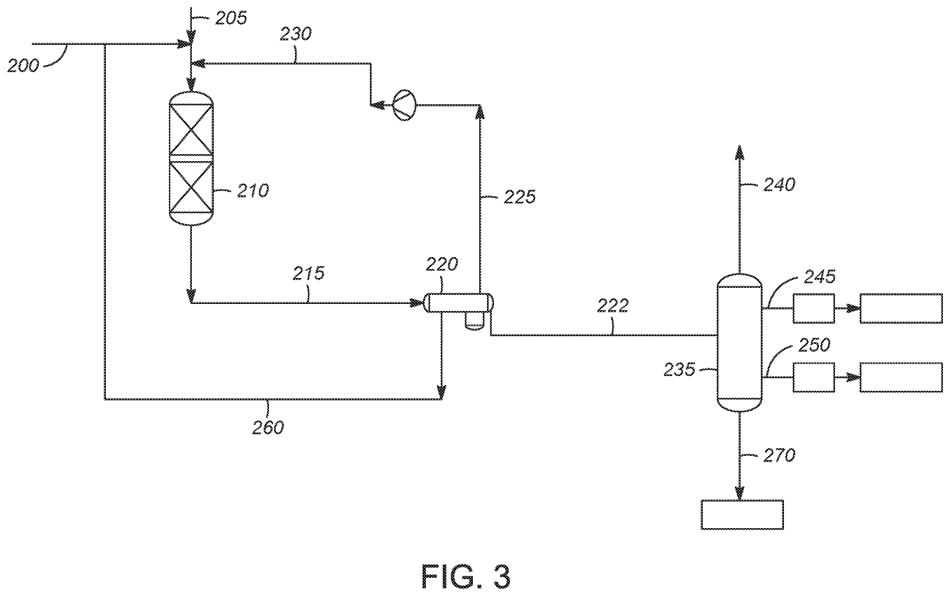

FIG. 3 shows an embodiment in which the hydrotreating and hydrocracking occur in sections within the same reactor. A feed 200 that has been treated to remove sulfur is combined with a supply of make-up hydrogen 205 to enter an upper portion of reactor 210 which has a low temperature catalyst in the upper portion of the reactor and a high temperature catalyst in the lower part of the reactor. The resultant stream 21 is sent to a separator 220 with a light hydrocarbon portion 260 returned to be combined with feed 200. A portion that contains the heavier hydrocarbons is sent in line 222 to column 235 to be separated into off gas stream 240, LPG stream 245 and LNAP stream 250 containing the linear C₁₀-C₁₃ products to be further reacted to make linear alkyl benzene. A stream 270 is sent to an isomerization reactor.

The following are several examples of the use of different catalysts to crack a paraffin into the desired C₁₀-C₁₃ paraffins.

### Example 1

A normal pentadecane (n-C15) feed was contacted with a 0.75% wt Pt on γ-alumina catalyst, with 0.75% wt Cl at conditions of 350° C., 500 psig, 1 h-1 WHSV, 10 H2/HC, 1 g catalyst. The reaction generated 40% n-C15 conversion. On carbon basis, selectivities were as follows: 0.79% methane, 39.50% C2-C8 normal paraffins, 38.08% C9-C12 normal paraffins, 10.91% C13 and C14 normal paraffins and 10.74% isomerized pentadecanes. The advantages seen were a low degree of methane production but there was low activity and high feed isomerization levels that may limit recycling.

### Example 2

A n-C15 feed was contacted with a catalyst consisting of 0.5% wt Ru on ZrO₂ at 245° C., 200 psig, 2 h⁻¹ WHSV, 25 H₂/HC (by moles), 0.5 g catalyst. The reaction generated 75% n-C₁₅ conversion. The selectivities on a carbon basis were C1 8.36%, C2 to C8 normal paraffins 34.65%, C₉ to C₁₂ normal paraffins 34.60%, C₁₃ and C₁₄ normal paraffins 21.94% and iC₁₅ 0.44%. The advantages found were high activity and low level of isomerization but there was higher production of methane than with the Pt-based catalyst.

### Example 3

A n-C₁₅ feed was contacted with a catalyst consisting of 0.1% wt Ru on ZrO₂ at 285° C., 500 psig, 1 hr⁻¹ WHSV, 25 H2/HC (by moles), 1 g catalyst. The reaction generated 98% n-C₁₅ conversion. The selectivities on a carbon basis were Cl 3.08%, C₂-C₈ normal paraffins 58%, C₉-C₁₂ normal paraffins 34.57%, C13-C14 normal paraffins 4.33% and no detectable isomerized C₁₅ or heavier products.

The Ru-based catalyst is sensitive to sulfur in the feed. In the examples, all clean parts were used in the testing. Previous testing with sulfur contaminated reactor yielded very low activity. Therefore, use of Ru based catalyst requires ˜<0.1 ppm, preferably <0.03 ppm S in feed to maintain activity.

### Example 4

Table 1 shows the experimental results from a feed containing 10 wt % n-C16 and 90 wt % nC₁₈ contacted with a number of different catalysts including catalysts containing Pt on alumina, Ni on alumina, NiO on clay, Ni on alumina dispersed on an inert core, Ni—Mo on alumina and Mo on alumina. Each individual catalyst testing was carried out in a 0.46″ ID once through plant having a Gas, Liquid Feed, Rx, Stripper and Product collection section. Reactor was loaded with the catalyst/SiC ratio 3. A sulfur adsorbent was placed in the pre-heat zone of the reactor to ensure the feed that enters the reactor has no sulfur in it. All catalysts were in-situ reduced in hydrogen at temperatures˜400° C. followed by inducting the catalyst with 90/10 nC₁₈/nC₁₆ blend. Using the same feed, under conditions outlined in Table 1 catalyst activity measurements were taken. In general, it preferred to maximize yield of normal C10-C13, maximize linearity and minimize methane byproduct level.

TABLE 1

| | | | | Ni/alumina | Ni—Mo on | Mo on |
|---|---|---|---|---|---|---|
| Cat comp. | Pt/alumina | Ni/alumina | NiO/clay | on inert core | alumina | alumina |
| Temp., ° C. | 325 | 315 | 350 | 315 | 400 | 370 |
| Pressure, bars | 35.41 | 7 | 35.72 | 7 | 34.5 | 7 |
| H₂/oil | 10400 | 5000 | 10000 | 5000 | 3170 | 5000 |
| C₁₄ + | 5% | 24% | 54% | 33% | 31% | 53% |
| conversion | | | | | | |
| H₂ | −0.1 | −1.8 | −4.09 | −3.2 | −1.82 | −0.986 |
| consumption | | | | | | |
| Total C10-13 | 4.23 | 23.57 | 22.8 | 49.3 | 29.3 | 79 |
| KMTA | | | | | | |
| produced | | | | | | |
| nC10-nC13 | 1.13 | 23 | 20.65 | 48 | 22 | 72 |
| KMTA | | | | | | |
| Linearity | 26 | 97.61 | 90.54 | 97.52 | 75 | 91 |
| (nC₁₀-C₁₃) | 0.08 | 37 | 14.82 | 40.6 | 36.6 | 57 |
| Selectivity | | | | | | |
| Byproduct | 0.206 | 31.82 | 68.757 | 60.43 | 0.6 | 0.87 |
| CH₄ KMTA | | | | | | |

### Specific Embodiments

While the following is described in conjunction with specific embodiments, it will be understood that this description is intended to illustrate and not limit the scope of the preceding description and the appended claims.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated. A first embodiment of the disclosure is a process for converting a bionrenewable feedstream to a C₁₀ normal to C₁₃ normal paraffin stream by first treating said biorenewable feedstream to remove sulfur to produce a sulfur-free feedstream and contacting said sulfur-free feedstream in a cracking reactor with a catalyst selected from Ru—ZrO₂, Pt—Al₂O₃, Ni—ZrO₂ and a Mo-containing catalyst or mixtures thereof. An embodiment of the disclosure is one, any or all of prior embodiments in this paragraph up through the first embodiment in this paragraph wherein the catalyst comprises Ru—ZrO₂ (0.1 wt %). An embodiment of the disclosure is one, any or all of prior embodiments in this paragraph up through the first embodiment in this paragraph wherein the catalyst comprises about 5-10 wt % Mo on alumina. An embodiment of the disclosure is one, any or all of prior embodiments in this paragraph up through the first embodiment in this paragraph wherein the Mo containing catalyst further comprises about 0.05-0.5 wt % Ni. An embodiment of the disclosure is one, any or all of prior embodiments in this paragraph up through the first embodiment in this paragraph wherein said biorenewable feedstream comprises carbon chains comprising C₁₆ to C₂₂ carbons. An embodiment of the disclosure is one, any or all of prior embodiments in this paragraph up through the first embodiment in this paragraph process of claim 1 wherein said biorenewable feedstream comprises carbon chains comprising C₁₆ to C₁₈ carbons. An embodiment of the disclosure is one, any or all of prior embodiments in this paragraph up through the first embodiment in this paragraph wherein the biorenewable feedstream is selected from triglycerides, fats, oils or greases. An embodiment of the disclosure is one, any or all of prior embodiments in this paragraph up through the first embodiment in this paragraph wherein the biorenewable feedstream undergoes an additional conversion process to an intermediate stream comprising normal paraffins and where at least a port of the intermediate stream comprising normal paraffins is converted to the C₁₀ normal to C₁₃ normal paraffin stream. An embodiment of the disclosure is one, any or all of prior embodiments in this paragraph up through the first embodiment in this paragraph wherein the C₁₀ to C₁₃ normal paraffin stream has over 80% linearity. An embodiment of the disclosure is one, any or all of prior embodiments in this paragraph up through the first embodiment in this paragraph wherein the C₁₀ to C₁₃ normal paraffin stream has over 95% linearity. An embodiment of the disclosure is one, any or all of prior embodiments in this paragraph up through the first embodiment in this paragraph wherein the normal paraffin stream has over 98% linearity An embodiment of the disclosure is one, any or all of prior embodiments in this paragraph up through the first embodiment in this paragraph further comprising treating the C₁₀ normal to C₁₃ normal paraffin stream to remove isomerized C₁₀ to C₁₃ hydrocarbons. An embodiment of the disclosure is one, any or all of prior embodiments in this paragraph up through the first embodiment in this paragraph wherein the sulfur is removed from the biorenewable feed stream by sending the biorenewable feedstream through an adsorption bed before being sent to be contacted with said catalyst. An embodiment of the disclosure is one, any or all of prior embodiments in this paragraph up through the first embodiment in this paragraph wherein the process produces less than 25 wt % methane. An embodiment of the disclosure is one, any or all of prior embodiments in this paragraph up through the first embodiment in this paragraph wherein said process produces less than 5% methane. An embodiment of the disclosure is one, any or all of prior embodiments in this paragraph up through the first embodiment in this paragraph wherein the feedstream is first sent to a hydrotreating reactor and then sent to a reactor and then the C₁₀-C₁₃ stream is sent to be converted to a linear alkyl benzene. An embodiment of the disclosure is one, any or all of prior embodiments in this paragraph up through the first embodiment in this paragraph wherein said catalyst in said hydrocracking reactor is partially reduced. An embodiment of the disclosure is one, any or all of prior embodiments in this paragraph up through the first embodiment in this paragraph wherein the cracking reactor is a combination of a hydrotreating and hydrocracking reactor.

1. A process for converting a biorenewable feedstream to a C₁₀ normal to C₁₃ normal paraffin stream by first treating said biorenewable feedstream to remove sulfur to produce a sulfur-free feedstream and contacting said sulfur-free feedstream in a cracking reactor with a catalyst selected from Ru—ZrO₂, Pt—Al₂O₃, Ni—ZrO₂ and a Mo-containing catalyst or mixtures thereof.

2. The process of claim 1 wherein said catalyst comprises Ru—ZrO2 (0.1 wt %).

3. The process of claim 1 wherein said catalyst comprises about 5-10 wt % Mo on alumina.

4. The process of claim 3 wherein said catalyst further comprises about 0.05-0.5 wt % Ni.

5. The process of claim 1 wherein said biorenewable feedstream comprises carbon chains comprising C₁₆ to C₂₂ carbons.

6. The process of claim 1 wherein said biorenewable feedstream comprises carbon chains comprising C₁₆ to C₁₈ carbons.

7. The process of claim 1 wherein said biorenewable feedstream is selected from triglycerides, fatty acids, fats, oils or greases.

8. The process of claim 1 wherein the biorenewable feedstream undergoes an additional conversion process to an intermediate stream comprising normal paraffins and wherein at least a portion of the intermediate stream comprising normal paraffins is converted to the C₁₀ normal to C₁₃ normal paraffin stream.

9. The process of claim 1 wherein said C10 to C13 normal paraffin stream has over 80% linearity.

10. The process of claim 1 wherein said C₁₀ to C₁₃ normal paraffin stream has over 95% linearity.

11. The process of claim 1 wherein said C₁₀ to C₁₃ paraffin stream has over 98% linearity.

12. The process of claim 1 further comprising treating said C10 normal to C13 normal paraffin stream to remove branched C10 to C13 hydrocarbons.

13. The process of claim 1 wherein said sulfur is removed from said biorenewable feed stream by sending said biorenewable feedstream or partially converted biorenewable feedstream through an adsorption bed before being sent to be contacted with said catalyst.

14. The process of claim 1 wherein said process produces less than 25 wt % methane.

15. The process of claim 1 wherein said process produces less than 5% methane.

16. The process of claim 1 wherein said feedstream is first sent to a hydrotreating reactor and then sent to a reactor to produce the C₁₀-C₁₃ normal paraffin feed stream and then said C₁₀-C₁₃ normal paraffin feed stream is sent to be converted to a linear alkyl benzene.

17. The process of claim 13 wherein said catalyst is partially reduced.

18. The process of claim 1 wherein said cracking reactor is a combination of hydrotreating and hydrocracking reactor.
